(19) United States
(12) Patent Application Publication
(10) Pub. No.: US 2021/0260306 A1
(43) Pub. Date: Aug. 26, 2021

(54) INTRAVENOUS FLUID WARMING SYSTEM

(71) Applicant: Vyaire Medical, Inc., Yorba Linda, CA (US)

(72) Inventors: Nikolaus GRAVENSTEIN, Gainesville, FL (US); Christopher VARGA, Laguna Hills, CA (US)

(21) Appl. No.: 16/478,099

(22) PCT Filed: Jan. 23, 2018
(86) PCT No.: PCT/US2018/014912

§ 371 Date: Jul. 15, 2019

Related U.S. Application Data
(60) Provisional application No. 62/449,979, filed on Jan. 24, 2017.

(51) Int. Cl. A61M 5/44; F24H 9/20; F24H 1/00; F24H 1/10

(52) U.S. Cl. CPC A61M 5/44; F24H 9/2014; F24H 1/0018; A61M 2205/3368; A61M 2205/52; A61M 2205/3334; F24H 1/101

(57) ABSTRACT

A fluid warming device has a heat exchange body having an input port and an output port and conducts fluid from the input port to the output port. The fluid warming device has a heater assembly configured to transfer heat to the heat exchange body. The fluid warming device may also have a temperature sensor for measuring a temperature of the heater assembly, and a power sensor for measuring a power to the heater assembly. The fluid warming device also has a controller connected to the temperature sensor and the power sensor. The controller calculates a fluid flow rate and a total volume of fluid delivered through the heat exchange body based on the temperature and the power.

### CROSS-REFERENCE TO RELATED APPLICATIONS

The present disclosure is the U.S. National Phase of International Application No. PCT/US2018/014912, filed Jan. 23, 2018, entitled, “INTRAVENEOUS FLUID WARMING SYSTEM,” which is related to and claims priority under 35 U.S.C. § 119(e) of U.S. Provisional Patent Application No. 62/449,979, entitled “INTRAVENOUS FLUID WARMING SYSTEM,” filed on Jan. 24, 2017.

### BACKGROUND

Intravenous (IV) fluid warming devices heat an IV fluid prior to introducing the fluid (e.g., crystalloids, colloids, blood products, drugs, etc.) into a patient. The rate and total amount of intravenous fluids of various types delivered to the patient may affect resuscitation, proper hydration, replacement of lost blood, maintenance and optimization of cardiac output and circulation, as well as the general health of body tissues and the prevention of edema and ischemia.

IV fluids may be delivered to patients by using a pump (e.g., an infusion pump) or a gravity-feed setup in which the IV fluid supply is elevated with respect to the patient's IV site, and the hydrostatic pressure difference drives the flow. In gravity-feed setups, which are in common perioperative use for delivery of crystalloids, colloids, and drug solutions, a flow rate and total volume of fluid may be estimated by a clinician. For example, the clinician may estimate the flow rate by estimating drips per unit of time and calculate the flow rate based on a drop volume provided on each IV set by the manufacturer. The total volume of fluid delivered may be estimated by viewing change in fluid level against graduations on IV bags. The clinician may then manually record the estimated values for the flow rate and/or total volume.

### SUMMARY

It may be advantageous for a fluid warming device to calculate the flow rate and the total volume of fluid delivered, and further to transmit the calculated values. One type of exemplary medical fluid warming system is described in U.S. Pat. No. 7,158,719, the disclosure of which is incorporated by reference herein. In this device, fluid passes along a generally serpentine fluid flow path through a removable/disposable heat exchange body. The heat exchange body is in thermal contact with a resistive film heater via thermally conductive layers interposed between the heat exchange body and the heater. Temperature sensors are provided that sense the temperature of the heat exchange body and of the heater.

According to some aspects, a fluid warming device may include a heat exchange body comprising an input port and an output port. The head exchange body is configured to conduct fluid from the input port to the output port. The fluid warming device may include a heater assembly configured to transfer heat to the heat exchange body. The fluid warming device may also include a temperature sensor configured to measure a temperature of the heater assembly, and a power sensor configured to measure a power to the heater assembly. The fluid warming device may also include a controller connected to the temperature sensor and the power sensor. The controller may be configured to determine a fluid flow rate through the heat exchange body based on the temperature and the power.

Some aspects provide that a fluid warming device may include a heat exchange body comprising an input port and an output port. The heat exchange body is configured to conduct fluid through the heat exchange body in one direction from an input port to an output port. The fluid warming device may include a housing configured to removably receive the heat exchange body. The fluid warming device may include a heater assembly disposed within the housing and configured to transfer heat to the heat exchange body. The fluid warming device may include a first slidable cover and a second slidable cover configured to hold the heat exchange body against the heater assembly. The fluid warming device may also include a temperature sensor configured to measure a temperature of the heater assembly, and a power sensor configured to measure a power to the heater assembly. The fluid warming device may include a controller connected to the temperature sensor and the power sensor. The controller may be configured to determine a fluid flow rate and a total volume of fluid delivered through the heat exchange body based on the temperature and the power.

According to aspects, a fluid warming device may include a heat exchange body comprising an input port and an output port. The heat exchange body is configured to conduct fluid from the input port to the output port. The fluid warming device may include a housing configured to removably receive the heat exchange body. The fluid warming device may include a heater assembly disposed within the housing and configured to transfer heat to the heat exchange body. The fluid warming device may also include a temperature sensor configured to measure a temperature of the heater assembly and a power sensor configured to measure a power to the heater assembly. The fluid warming device may also include a controller connected to the temperature sensor and the power sensor. The controller may be configured to determine a fluid property of the fluid, determine a temperature difference between the input port and the output port, continuously measure the power, and determine a fluid flow rate based on the fluid property, the temperature difference, and the measured power.

### BRIEF DESCRIPTION OF THE DRAWINGS

The disclosure will be more fully understood from the following detailed description taken in conjunction with the accompanying drawings in which:

FIG. 1 is a block diagram of a fluid warming device according to aspects of the present disclosures;

FIG. 2 is an isometric view of a fluid warming device illustrating slidable covers in a closed position according to aspects of the present disclosures;

FIG. 3 is an isometric view of the fluid warming device of FIG. 2 illustrating the slidable covers in a half closed position according to aspects of the present disclosures;

FIG. 4 is an isometric exploded view of the fluid warming device of FIG. 2 with the slidable covers in an open position and a disposable set removed according to aspects of the present disclosures;

FIG. 5 is an isometric view of a main body of the housing of the device of FIG. 2 with the slidable covers fully removed according to aspects of the present disclosures;

FIG. 6 is a plan view of the device of FIG. 3;

FIG. 7 is a cross sectional view taken along line A-A of FIG. 6;

FIG. 8 is a schematic view of a disposable set and heater assembly of the fluid warming device of FIG. 2;

FIG. 9 is a side view of a fluid warming device illustrating gripping faces on the slidable covers according to aspects of the present disclosures;

FIG. 10A is a flow chart illustrating a system for determining a fluid flow rate according to aspects of the present disclosures;

FIG. 10B is a flow chart illustrating a system for identifying a fluid type according to aspects of the present disclosures;

FIG. 11 is a graph illustrating a linear best fit of flow rate versus heater current for Lactated Ringers according to aspects of the present disclosures; and

FIG. 12 is a graph illustrating a comparison between heater current versus flow rate for Plasmalyte® with heater current versus flow rate for Lactated Ringers.

### DETAILED DESCRIPTION

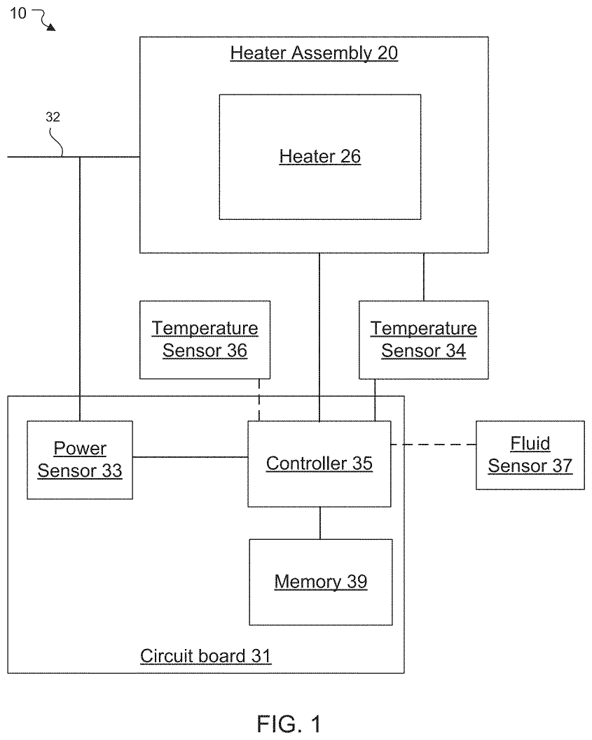

A fluid warming device or warmer 10 according to the present disclosure is illustrated in FIG. 1. The fluid warming device 10 includes a heater assembly 20, which includes a heater 26. A power line 32 to the heater assembly 20 provides power from a suitable power source. A power sensor 33 is coupled to the power line 32 and is configured to measure a power of the power line 32. A temperature sensor 34 measures temperature of the heater assembly 20, for example to measure or estimate a temperature of fluid flowing out of the fluid warming device 10. Certain implementations may include a temperature sensor 36, which may be configured to measure or estimate a temperature of fluid flowing into the fluid warming device 10. Certain implementations may include a fluid sensor 37, which may be configured to identify the fluid or type of fluid flowing into the fluid warming device 10. A controller 35 is coupled to the heater assembly 20, power sensor 33, temperature sensor 34, and in certain implementations the temperature sensor 36 and the fluid sensor 37. The controller 35 may be one or more processors. A memory 39 is coupled to the controller 35 and may store values, such as fluid heat capacities, used by the controller 35. The memory 39 may also store relations of fluid flow rate as a function of power or current. The controller 35 and the memory 39 may be disposed on a circuit board 31 in the fluid warming device 10. In certain implementations, the power sensor 33 may be disposed on the circuit board 31. The controller 35 is configured to control the heating operation of the heater assembly 20 as well as determine a fluid flow rate and a volume of fluid delivered, as will be discussed further below.

The power of the power line 32 may be measured by the power sensor 33, which may comprise, for example, a wattmeter circuit. In a direct current (DC) power system, power (watts) is proportional to current (amps) and voltage (volts), such that power consumption is determined from the product of measured current and measured voltage. In an alternating current (AC) power system, power (watts) is proportional to current (amps), voltage (volts), and a power factor (ratio of real power to apparent power, and corresponds to a phase difference between current and voltage waveforms). Power consumption is determined by integrating the product of the current, voltage, and power factor over time. In implementations in which voltage is constant (e.g., either on or off for periods of time as needed for heating), the power may be measured by measuring current such that the power sensor 33 is a current sensor. In implementations in which heater power is modulated by changing voltage, the power may be determined by measuring current and voltage. In such implementations, rather than having a sensor for measuring the voltage, the voltage may be known by the controller 35 which modulates the voltage such that the power sensor 33 is a current sensor.

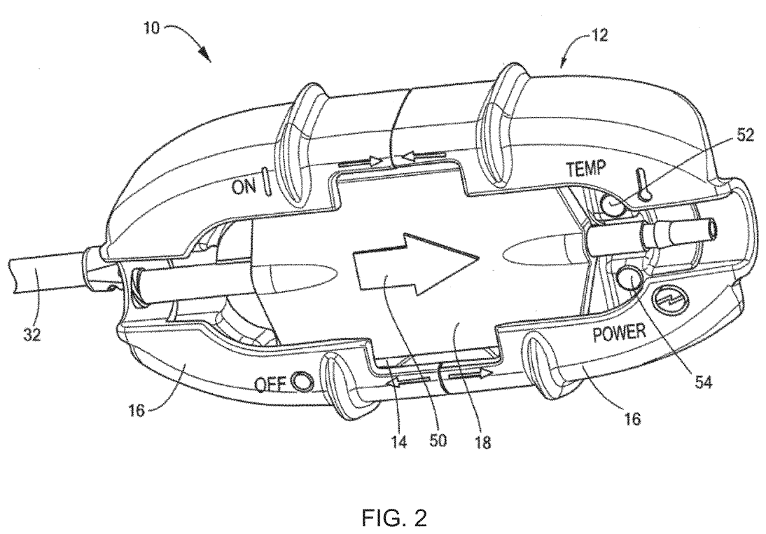

An embodiment of the fluid warming device 10 is illustrated in FIGS. 2-8. The fluid warming device 10 includes a housing 12 having a main body 14 and two sliding or slidable covers 16. Within the housing 12, supported by the main body, are a removable heat exchange body 18 and a heating or heater assembly 20. The sliding covers 16 are independently slidable to a closed position in which they retain the removable heat exchange body 18 in place, as described more fully below. The slidable covers 16 are preferably identical.

The removable heat exchange body 18 and the heating assembly 20 are illustrated schematically in FIG. 8. The heat exchange body, also called a disposable or removable set, includes an input port or connector 22 connectable to an IV tubing line from a source of IV fluid, which may include an infusion pump. The disposable set also includes an output port or connector 24 connectable to a further IV tubing line to deliver the IV fluid to the patient. Within the disposable set, the IV fluid flows along a flow path (not shown) having a serpentine or other suitable configuration between the input and output ports to optimize heat transfer to the fluid. See, for example, U.S. Pat. No. 7,158,719 incorporated by reference in its entirety. The disposable set 18 is formed from any suitable material, such as aluminum, to facilitate heat transfer to the fluid flowing therein. When inserted in the housing 12 with the sliding covers 16 in a closed position, the disposable set 18 is held in thermal contact with the heater assembly 20, so that heat transfer from the heater assembly 20 to the disposable set 18 causes heating of an IV fluid flowing therethrough.

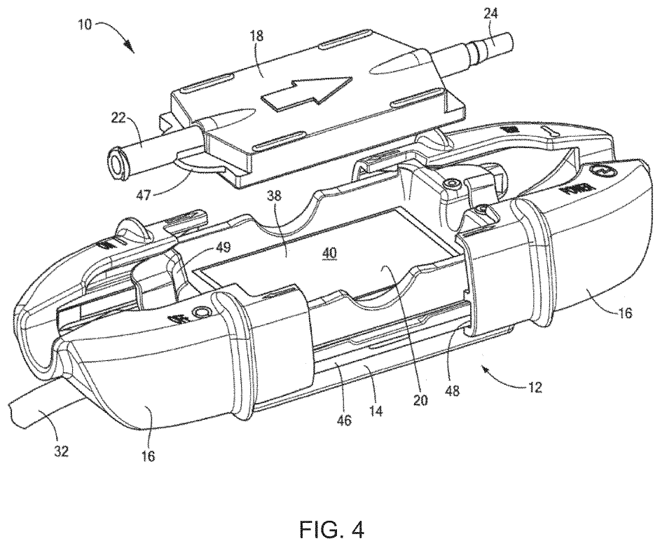

The heater assembly 20 is affixed within the main body 14 of the housing 12. The heater assembly 20 includes a heater 26 and one or more thermally conductive layers 28, 30 interposed between the disposable set 18 and the heater 26. Preferably, the heater 26 is an electrically powered resistive thin film heater. A power line 32 to the heater from a suitable power source is provided. Alternatively, the device may include a battery compartment or a connection to a battery pack, for example, for portable operation. Temperature sensors 34, 36 are provided that sense the temperature of the disposable set 18 and of the heater 26. The thermally conductive layers also electrically insulate the disposable set from the resistive heater 26. One thermally conductive layer 28 may suitably comprise a phase transition material, and the other thermally conductive layer 30 may suitably comprise a material such as a graphite to optimize heat transfer between the heater and the disposable set. It will be appreciated that other or further thermally conductive layers may be provided. As seen in FIGS. 4 and 8, the main body 14 includes a compartment 38 on one side to receive the disposable set 18 in contact with an exposed surface 40 of the uppermost thermally conductive layer 30.

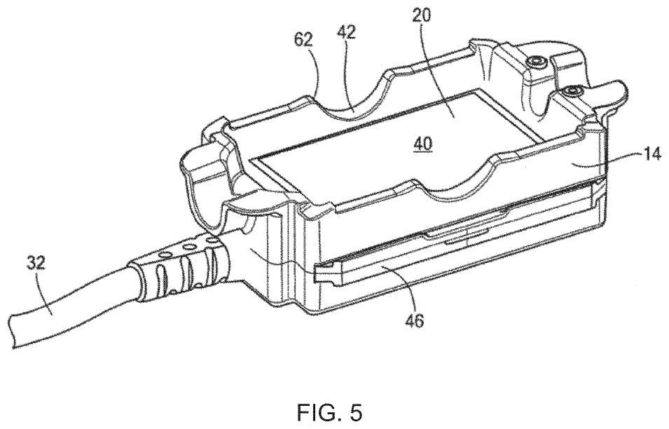

As noted above, the heat exchange body or disposable set 18 is removable from the housing 12. The disposable set 18 can be removed from the main body 14 of the housing 12 by sliding the two opposed sliding covers 16 outwardly in opposite directions. In this manner, the removable set 18 can be lifted out of the housing 12 with the IV tubing still attached to the input and output connectors 22, 24, without breaking the fluid path. Finger cutouts 42 may be provided for ease of grasping the disposable set 18 in the main body 14, as seen in FIG. 5.

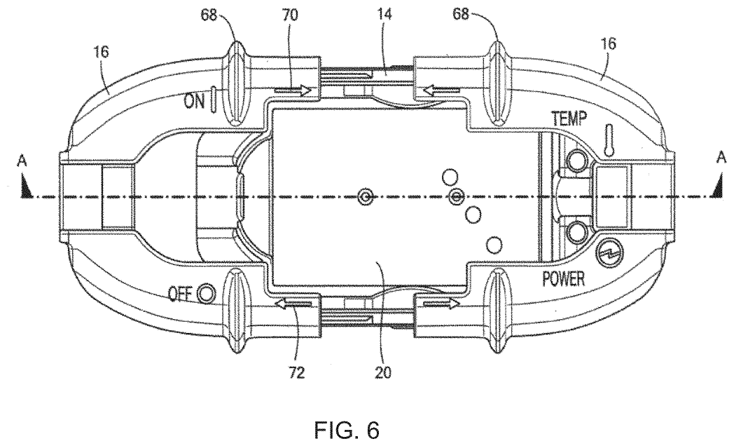

Any suitable sliding mechanism to allow the covers 16 to move axially into the closed position can be provided. In embodiments shown in FIGS. 5 and 6, the main body 14 of the housing 12 includes protruding longitudinal tracks 46 along two opposed longitudinal outer wall surfaces of the main body 14. The sliding covers 16 include complementary longitudinal recesses 48 along inner wall surfaces that mate with the tracks 46 and allow the covers to slide axially along the main body, as seen in FIG. 4. When in the closed position, the sliding covers 16 extend over the edges of the disposable set 18 within the recess 48 of the main body, thereby retaining the disposable set therein (shown in FIG. 2). The covers 16 also compress the disposable set 18 to the outermost thermally conducting surface 40 of the heater assembly. This compression provides the necessary pressure for proper heat transfer between the heater assembly 20 and the disposable set 18. The covers 16 may be retained in the closed position by frictional engagement with the disposable set 18. Alternatively, any suitable latching or retaining mechanism may be provided.

Also, the covers 16 may not block the view of the bulk of the mid portion of the disposable set 18, allowing the operator to view the fluid passing through the disposable set. The disposable set 18 is also keyed to the main body 14 in any suitable manner so that it fits within the compartment 38 in the correct orientation. For example, in FIG. 4, one end 47 of the disposable set 18 may be rounded to fit within a correspondingly rounded portion 49 of the compartment 38. The disposable set 18 may include an arrow 50 thereon, seen in FIG. 2, to provide an indication of the direction of flow, so that the disposable set 18 is inserted in the housing 12 in the correct orientation. The covers 16 do not block this arrow. Also, the main body 14 preferably includes indicator lights, such as LEDs, thereon. For example, one LED 52 may provide an indication of temperature at the output port 24, and another LED 54 may provide an indication that the heater 26 is connected to the power source. The covers 16 do not block these indicator lights 52, 54 either.

In one embodiment, the covers 16 can be maintained in two positions on the main body 14 or can be removed fully from the main body 14. While on the main body 14, the covers 16 can be in a fully closed position, as in FIG. 2, or an open position, as in FIG. 4. The covers 16 can include magnets or Hall effect devices or other proximity sensors that interface with a corresponding component within the main body 14 to determine the positions of the covers and cause operation of any appropriate switches. In a further embodiment, the covers 16 can be maintained in a third or intermediate, half-closed, position on the main body 14, described further below.

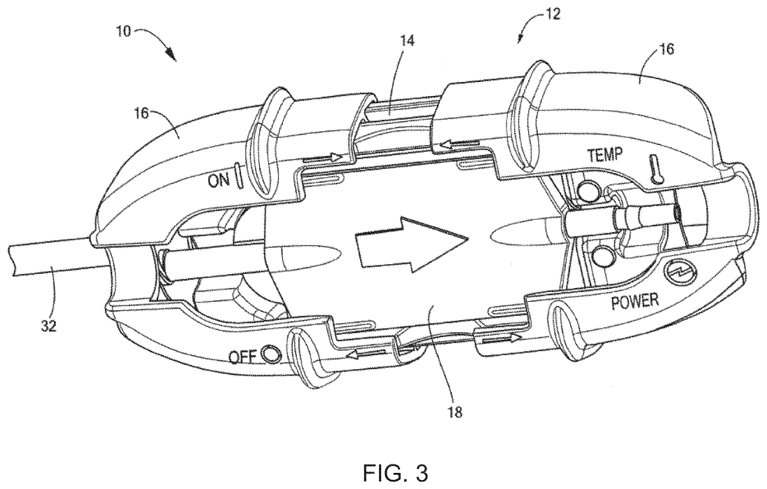

More particularly, in the fully closed position, (see FIG. 2), the covers 16 apply full pressure to the disposable set 18 to ensure good thermal contact with the heater assembly 20. In this position, the sliding covers 16 can also be used to turn the power on to commence warming and/or to activate any audible or visible alarm(s). In the half closed position (see FIG. 3), the disposable set 18 is still held in place by the covers 16, but warming is stopped, the audible alarm is silenced, and the visual indicators 52, 54 are turned off. The status LED 54 could be flashed in battery operation to inform the user that the warmer is connected to the battery and draining. When the covers 16 are in the open position (see FIG. 4), the disposable set 18 can be inserted and removed. No heating takes place, the audible alarm is silenced, and visual indicators 52, 54 are turned off. The status LED 54 could be flashed in battery operation to inform the user that the heater is 26 connected to the battery and draining.

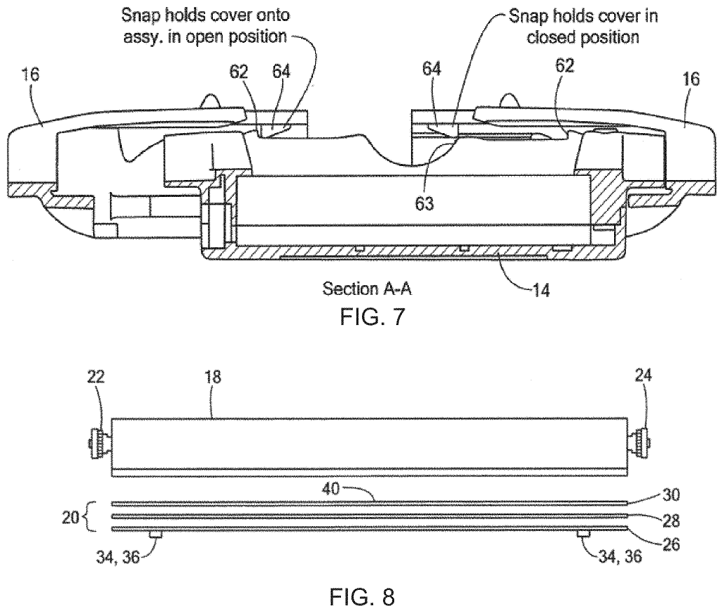

Any suitable latching or retaining mechanism can be provided to retain the covers 16 in the desired positions relative to the main body 14. For example, as shown in FIGS. 6 and 7, recessed surfaces 62 are provided on the main body 14 that latch with corresponding tabs 64 on the covers 16 in the open position, preventing the covers 16 from readily coming off the main body 14. Also, the tabs 64 abut surfaces 63 to hold the covers 16 in the closed position. Finger grips 68 are provided to aid in grasping the covers 16 to push or pull them to the desired position. The closed (and power on) position can be indicated by arrows 70 and an adjacent “ON” marking on the covers. Similarly, the open (and power off) position can be indicated by arrows 72 and an adjacent “OFF” marking on the covers 16. The covers 16 can be fully removed from the main body 14 in any suitable manner, for example, by the insertion of a suitable tool, such as a screw driver or dime, to lift the tab 64 over the surfaces 62. Alternatively, a latching or retaining mechanism can be configured to release simply by the use of sufficient force. Removal of the covers 16 allows the device to be readily cleaned. Alternatively, passageways in the interior surfaces of the covers 16 and a water-tight main body housing allow cold sterilization by dipping in a sterilization fluid without complete removal of the covers 16.

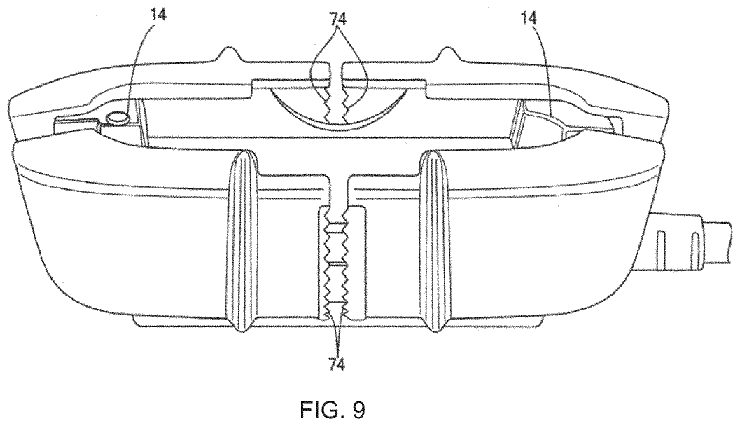

Referring to FIG. 9, the sliding covers 14 may include opposed faces 74 that include gripping teeth thereon to form gripping faces. The gripping faces can be used to grip hospital clothing or bedding and hold the warmer 10 in place to reduce stress on the IV line when the covers are fully closed.

The controller 35 may control or regulate the amount of heat delivered to the IV fluid using feedback loops based on fluid temperature measurements. The amount of heat delivered per unit time may be modulated by turning the heater on and off at a rate proportional to how close the actual temperature of the fluid is to a target value. Alternatively, the actual heater may be continuously energized at a level proportional to how close the actual temperature of the fluid is to the target value. The amount of heating power provided to reach a given temperature is proportional to the fluid flow rate, the difference between the target temperature and the initial fluid temperature, and the properties of the IV fluid itself, according to the heat transfer equation:

Q=m·Cₚ·ΔT. (1)

Wherein Q is heat transferred per unit time (J/s), m is the mass flow rate of the fluid (kg/s), cₚ is the heat capacity of the fluid (J/Kg−K) and ΔT is the difference between the initial and final temperature of the fluid, Tₒᵤₜₗₑₜ−Tᵢₙₗₑₜ (K).

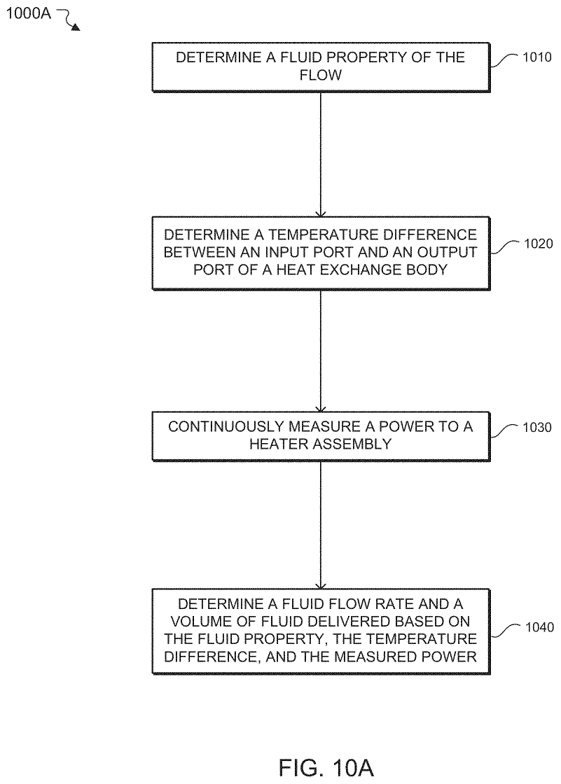

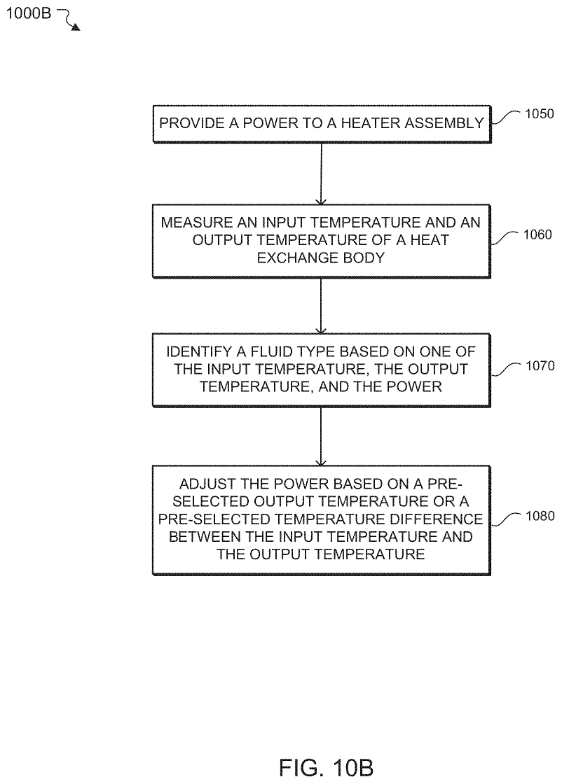

FIG. 10A shows a flowchart 1000A of a process of determining fluid flow rate and volume of fluid delivered, according to aspects. At 1010, the controller 35 determines a fluid property of the flow through the fluid warming device 10. The fluid property may correspond to the heat capacity of the fluid. The fluid property may be predetermined or preset, for example stored in the memory 39.

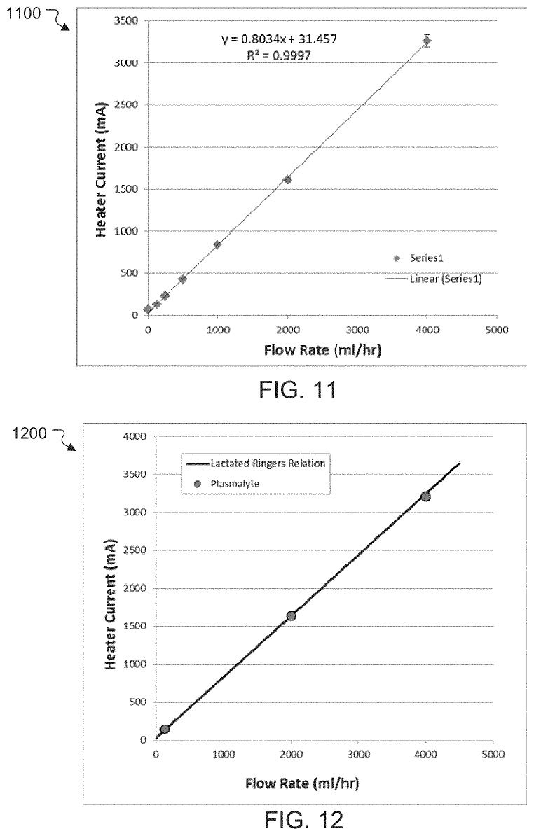

Referring to FIG. 11, a graph 1100 shows a measured heater current as a function of the flow rate of a common intravenous crystalloid solution, Lactated Ringers. As seen in the graph 1100, the fluid property may be represented by a linear equation, which the controller 35 may use to calculate the flow rate.

Referring to FIG. 12, a graph 1200 shows a comparison of heater current versus flow at three points for another common crystalloid, Plasmalyte® to the heater versus current relation for Lactated Ringers. As seen in FIG. 12, the Lactated Ringers relation approximates that of the Plasmalyte®. Therefore, in certain implementations, a single fluid property, which may be represented by an equation, may be applied to all crystalloid fluids.

In certain other implementations, the memory 39 may store multiple fluid properties, corresponding to different fluids or types of fluids. The fluid sensor 37 may detect a fluid or type of fluid flowing through the heat exchange body 18 such that the controller 35 selects the corresponding fluid property stored in the memory 39. The fluid sensor 37 may be a diode or optical sensor capable of identifying fluid or type of fluid based on temperature, diffraction, reflection, or transmission of electromagnetic radiation. Alternatively, the fluid sensor 37 may be another sensor capable of identifying fluids, for example through electrical conductivity, heat sensitivity, etc. In other implementations, the user may input a fluid type to the controller 35, or the fluid sensor 37 may be a scanner, such as a bar code scanner or radio frequency identification (RFID) reader, which can be used to read fluid type from an IV bag or other fluid supply (e.g. syringe, bottle, etc.).

In certain implementations, for example implementations without the fluid sensor 37, the controller 35 may be configured to identify the fluid type based on the temperature sensors 36 and/or 34 or the power sensor 33. Crystalloids are commonly stored at room temperature and are similar enough to use a single fluid property for all crystalloid fluids. However, blood products, such as blood or fresh frozen plasma, are generally colder than room temperature. For example, blood products may be stored at 4 degrees C., which may be a 10-20 degree difference from crystalloids. Cold blood products may also be diluted with equal volumes of crystalloid prior to use, resulting in a mixture temperature which may be 5-10 degrees different from crystalloids. Thus, the controller 35 may identify a blood product based on a low input temperature (e.g., lower than ambient temperature), or a high power requirement (e.g., more power required to heat a fluid starting at below ambient temperature) for heating the blood product. In other words, the controller 35 may determine the fluid property to use, based on detecting a low input temperature or a high power demand. For example, the controller 35 may detect the low input temperature as the temperature at the input port 22 being lower than a threshold temperature, which may correspond to the ambient temperature. The controller 35 may detect the high power demand based on comparing the power demand to a threshold power demand, which may correspond to a power demand for heating the fluid 10-15 degrees more (from the ambient temperature) to reach a target temperature.

Returning to FIG. 10A, at 1020, the controller 35 determines a temperature difference between the input port 22 and the output port 24 of the heat exchange body 18. In certain implementations, the temperature at the input port 22 may be represented by an ambient or room temperature stored in the memory 39 (for example a default value such as 20 degrees C. or input by a user), or may be measured by the temperature sensor 36. The temperature sensor 36 may be external to the fluid warming device 10, for example a remote temperature sensor, which may be mounted to an IV bag or mounted in a thermal well connected to a thermal mass which simulates temperature of the fluid being slightly colder than ambient temperature. The temperature sensor 36 may alternatively be an infrared sensor near the input port 22, or may be a thermistor on the heater 26, as described above.

The temperature at the output port 24 may be measured by the temperature sensor 34, which may be a thermistor on the heater 26, as described above. Alternatively, the temperature sensor 34 may be an infrared sensor or other temperature sensor located near the output port 24. In other implementations, the temperature at the output port 24 may be determined by temperature sensors on the heater 26.

At 1030, the power sensor 33 continuously monitors a power to the heater assembly 20. As stated above, the power sensor 33 may be in the fluid warming device 10, for example on the circuit board with the controller 35, or may be external to the fluid warming device 10. The power sensor 33 measures power in the power line 32. Power relates to current and voltage. Thus, in certain implementations in which a constant voltage is applied for heating, current may be measured and the calculations described below may use power derived from the current, using the known constant voltage. In such implementations, the power sensor 33 may be a current sensor.

At 1040, the controller 35 determines a fluid flow rate and a volume of fluid delivered based on the fluid property, the temperature difference, and the measured power. Based on the heat transfer equation, the mass flow rate (m) may be calculated using the measured power converted to heat (Q), the temperature difference (ΔT), and the heat capacity for the fluid (cₚ). For example, the fluid property described above may be an equation in which the measured values may be input to calculate the flow rate. Based on the flow rate, the volume of fluid may be determined. For example, by continuously measuring power and integrating the fluid property equation over a period of time, the volume may be calculated. The period of time may be total time, for example from the start of IV infusion, or may be a predetermined time period, such as the last hour, or other time period selected by the user.

The fluid warming device 10 may be communicatively coupled to an interface, which allows input of parameters as described herein, and may further display the calculated values, such as the flow rate and volume of fluid delivered. The fluid warming device 10 may also be configured to communicate the calculated values. For example, the fluid warming device 10 may be configured to communicate the fluid flow rate and the volume of fluid delivered to an electronic medical record (EMR) system.

FIG. 10B is a flow chart illustrating a method 1000B for identifying a fluid type in the fluid warming device 10, according to aspects of the present disclosure. Accordingly, step 1050 includes providing a power to the heater assembly. Step 1060 includes measuring an input temperature and an output temperature of the heat exchange body in the fluid warming device 10. Step 1070 includes identifying the fluid type flowing through the fluid warming device based on one of the input temperature, the output temperature, and the power. And step 1080 includes adjusting the power based on a pre-selected value of the output temperature or a pre-selected temperature difference between the input temperature and the output temperature.

In some embodiments, 1080 includes modulating the heat provided to the fluid warming device 10. Power to the heater 26 can be increased or decreased to adjust the fluid temperature to ensure that the fluid is at an appropriate temperature when it reaches the patient. More particularly, some IV fluids that have been warmed are administered at very low flow rates. These fluids cool as they travel down the IV tubing to the patient. The greater the difference between ambient temperature and the fluid temperature, the greater the heat losses from the IV fluid to the ambient environment.

The controller 35 performs the calculations and communicates with the heater 26 to make the desired adjustments. Heater power is determined by the difference between a target temperature (typically in the range of 39 to 41 degrees C.), and the actual fluid temperature.

The controller 35 calculates the temperature drop across the heat exchanger 18. The temperature drop is equal to the heater power multiplied by the thermal resistance of the heater assembly 20. The thermal resistance can be readily determined from the thickness, thermal conductivity and area of the materials between the heater 26 and the fluid and stored as a constant, which may be stored in the memory 39.

Then, the controller 35 calculates the temperature loss of the IV tubing to the environment. First, the difference between the fluid target temperature and the ambient temperature is determined. The temperature loss may be due to conductive, convective, and radiative heat losses. The ambient temperature is measured by a suitable sensor, such as the temperature sensor 36 which may be located within the warming device 10 in close contact with the housing, which is very close to ambient temperature. Alternatively, the ambient temperature may be measured by the temperature sensor 36 which may be located outside of the fluid warming device 10. In other implementations, the ambient temperature may be represented by a value stored in the memory 39, which may have been previously entered. The heat losses from the tubing may be derived from experimentation with various lengths of the IV tubing and various flow rates, and may be stored in the memory 39.

The controller may determine if the IV tubing heat losses are greater than a threshold, such as 1 degree C. The controller also determines if the total drop along the IV tubing and across the heat exchanger 18 is greater than a drop limit. The drop limit is the maximum temperature that the fluid can be artificially raised so that the allowable surface temperature on the heat exchanger is not exceeded, for example, no greater than 3 degrees C. from the desired target temperature. If the IV tubing loss is greater than 1 degree and the total drop along the IV tubing across the heat exchanger 18 is greater than the drop limit, the actual fluid temperature is calculated as the measured fluid output temperature minus the drop limit. Otherwise, the actual fluid temperature is calculated as the fluid output temperature in the IV tubing drop minus the IV tubing drop minus the heat exchanger drop. Using the calculated value of the actual temperature, heater power is adjusted appropriately.

The disclosure is not to be limited by what has been particularly shown and described, except as indicated by the appended claims.

The term “machine-readable storage medium” or “computer-readable medium” as used herein refers to any medium or media that participates in providing instructions or data to controller 35 for execution. The term “storage medium” as used herein refers to any non-transitory media that store data and/or instructions that cause a machine to operate in a specific fashion. Such a medium may take many forms, including, but not limited to, non-volatile media, volatile media, and transmission media. Non-volatile media include, for example, optical disks, magnetic disks, or flash memory. Volatile media include dynamic memory. Transmission media include coaxial cables, copper wire, and fiber optics. Common forms of machine-readable media include, for example, floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH EPROM, any other memory chip or cartridge, or any other medium from which a computer can read. The machine-readable storage medium can be a machine-readable storage device, a machine-readable storage substrate, a memory device, a composition of matter effecting a machine-readable propagated signal, or a combination of one or more of them.

As used in this specification of this application, the terms “computer-readable storage medium” and “computer-readable media” are entirely restricted to tangible, physical objects that store information in a form that is readable by a computer. These terms exclude any wireless signals, wired download signals, and any other ephemeral signals. Storage media is distinct from but may be used in conjunction with transmission media. Transmission media participates in transferring information between storage media. For example, transmission media includes coaxial cables, copper wire and fiber optics. Transmission media can also take the form of acoustic or light waves, such as those generated during radio-wave and infra-red data communications. Furthermore, as used in this specification of this application, the terms “computer,” “server,” “processor,” and “memory” all refer to electronic or other technological devices. These terms exclude people or groups of people. For the purposes of the specification, the terms display or displaying means displaying on an electronic device.

What is claimed is:

1. A fluid warming device, comprising:
a heat exchange body comprising an input port and an output port and configured to conduct fluid from the input port to the output port;
a heater assembly configured to transfer heat to the heat exchange body;
a temperature sensor configured to measure a temperature of the heater assembly;
a power sensor configured to measure a power to the heater assembly;
a controller connected to the temperature sensor and the current sensor, the controller configured to determine a fluid flow rate through the heat exchange body based on the temperature and the power.

2. The fluid warming device of claim 1, wherein the controller is further configured to determine a total volume of fluid delivered through the heat exchange body based on the temperature and the power.

3. The fluid warming device of any one of claims 1 and 2, wherein the power is measured by measuring a current to the heater assembly.

4. The fluid warming device of any one of claims 1 through 3, wherein the controller is configured to determine the fluid flow rate based on a fluid property, a temperature difference, and the power.

5. The fluid warming device of claim 4, wherein the controller is configured to identify the fluid in the heat exchange body, wherein the fluid property is determined based on the identified fluid.

6. The fluid warming device of claim 5, wherein the controller is configured to identify the fluid based on a temperature at the input port.

7. The fluid warming device of any one of claims 5 and 6, wherein the controller is configured to identify the fluid based on a power demand.

8. The fluid warming device of any one of claims 4 through 7, wherein the temperature difference is based on the temperature and a second temperature.

9. The fluid warming device of claim 8, wherein the first temperature sensor is disposed near the output port and a second temperature sensor is disposed near the input port to measure the second temperature.

10. A fluid warming device, comprising:
a heat exchange body comprising an input port and an output port and configured to conduct fluid therethrough in one direction from an input port to an output port;
a housing configured to removably receive the heat exchange body;
a heater assembly disposed within the housing and configured to transfer heat to the heat exchange body;
a first slidable cover and a second slidable cover configured to hold the heat exchange body against the heater assembly;
a temperature sensor configured to measure a temperature of the heater assembly;
a power sensor configured to measure a power to the heater assembly; and
a controller connected to the temperature sensor and the power sensor, the controller configured to determine a fluid flow rate and a total volume of fluid delivered through the heat exchange body based on the temperature and the power.

11. The fluid warming device of claim 10, wherein the controller is configured to determine the fluid flow rate based on a fluid property stored in a memory coupled to the controller, a temperature difference, and the power.

12. The fluid warming device of claim 11, further comprising a sensor configured to identify the fluid in the heat exchange body, wherein the fluid property is determined based on the identified fluid.

13. The fluid warming device of any ne of claims 11 and 12, wherein the temperature difference is based on the temperature and a second temperature.

14. The fluid warming device of claim 13, wherein the second temperature is based on an ambient temperature.

15. The fluid warming device of any one of claims 13 and 14, wherein the second temperature is measured by a second temperature sensor.

16. The fluid warming device of claim 15, wherein the first temperature sensor is disposed near the output port and the second temperature sensor is disposed near the input port.

17. A fluid warming device, comprising:
a heat exchange body comprising an input port and an output port and configured to conduct fluid from an input port to an output port;
a housing configured to removably receive the heat exchange body;
a heater assembly disposed within the housing and configured to transfer heat to the heat exchange body;
a temperature sensor configured to measure a temperature of the heater assembly;
a current sensor configured to measure a current to the heater assembly; and
a controller connected to the temperature sensor and the current sensor, the controller configured to:
determine a fluid property corresponding to the fluid;
determine a temperature difference between the input port and the output port;
continuously measure the current; and
determine a fluid flow rate based on the fluid property, the temperature difference, and the measured current.

18. The fluid warming device of claim 17, further comprising a sensor configured to identify the fluid in the heat exchange body, wherein the fluid property is determined based on the identified fluid.

19. The fluid warming device of claims 17 and 18, wherein the temperature difference is based on the temperature and a second temperature.

20. The fluid warming device of any one of claims 17 through 19, wherein the second temperature is measured by a second temperature sensor disposed near the input port, and the first temperature sensor is disposed near the output port.

21. The fluid warming device of any one of claims 17 through 20, further comprising a circuit board, wherein the controller and the current sensor are disposed on the circuit board.

22. The fluid warming device of any one of claims 17 through 21, wherein the controller is further configured to determine a total volume of fluid delivered through the heat exchange body based on the fluid property, the temperature difference, and the measured current.
